(19) United States
(12) Patent Application Publication
(10) Pub. No.: US 2009/0249503 A1
(43) Pub. Date: Oct. 1, 2009

(54) ENZYME CONJUGATES FOR USE AS DETOXIFYING AGENTS

(75) Inventor: Mary S. Rosendahl, Broomfield, CO (US)

Correspondence Address: SHERIDAN ROSS PC, 1560 BROADWAY, SUITE 1200, DENVER, CO, 80202, US

(73) Assignee: BOLDER BIOTECHNOLOGY, INC., Wheatridge, CO (US)

(21) Appl. No.: 11/721,024

(22) PCT Filed: Dec. 6, 2005
(86) PCT No.: PCT/US05/44273

§ 371 Date: Dec. 30, 2008

Related U.S. Application Data
(60) Provisional application No. 60/633,745, filed on Dec. 6, 2004.

(51) Int. Cl. A01K 67/00; C12N 9/16; C07H 21/00; C12N 5/00; C12N 5/06; C12N 1/20; A61P 39/00; A01H 5/00

(52) U.S. Cl. 800/13; 435/196; 536/23.2; 435/325; 435/348; 435/252.3; 435/254.2; 800/298

(57) ABSTRACT

Disclosed are detoxifying enzyme conjugates, including conjugates of variants of such detoxifying enzymes. The detoxifying enzymes are preferably chlolinesterases, and more preferably, butyrylcholinesterase. Also disclosed are methods of making and using such conjugates.

### FIELD OF THE INVENTION

The present invention generally relates to variants and conjugates of detoxifying enzymes and methods of making and using such variants and conjugates.

### BACKGROUND OF THE INVENTION

Exposure to organophosphorus (OP) compounds in the form of nerve agents is an ever-increasing military and civilian threat. OPs were first developed in the 1930's for use as insecticides. Their potency was recognized during World War II, and they were developed as nerve agents for use in chemical warfare. The acute toxicity of OPs is usually attributed to their irreversible inhibition of acetylcholinesterase (AChE). This results in the accumulation of acetylcholine at cholinergic synapses, particularly in the brain and diaphragm, producing a crisis that can culminate in death by respiratory failure. Current treatment for OP poisoning consists of a prophylatic dose of a spontaneously reactivating AChE inhibitor to protect ACHE from irreversible inhibition of OPs and post-exposure dose of anticholinergic drugs such as atropine sulfate, to counteract the effects of excess acetylcholine. Although these treatment regimens are effective in preventing lethality from OP poisoning, they do not prevent the post-exposure incapacitation, convulsions, performance problems and even permanent brain damage. In addition, the timing of the administration of these drugs is critical to achieving maximal therapeutic benefit. Accordingly, the need still exists for more effective preventative agents to combat the toxic effects of OP agents.

A number of enzymes exist in nature that are capable of detoxifying organophosphorus (OP) nerve agents, various pesticides and certain drugs of abuse such as cocaine or heroin. Carboxyl/cholinesterases (CE/ChEs) are a multi-gene family of enzymes that in vivo hydrolyze a diverse range of carboxylesters. Members of this family include the human carboxylesterase 1 (hCE1) and 2 (hCE2), acetylcholinesterase (ACHE) and butyrylcholinesterase (BuChE). A second family of enzymes that are able hydrolyze nerve agents are the phosphoric triester hydrolase enzymes such as organophosphorus hydrolase (OPH) (Dumas et al., 1989) and organophosphorus acid anhydrolase (OPAA) (Cheng, 1993; DeFrank, 1991). A third family of enzymes are the paraoxonase/arylestereases (PON1, PON2 and PON 3). These enzymes are similar to the carboxylesterases but differ in having cysteine rather than serine as the key component of their active centers.

Therefore, one concept for dealing with nerve agent toxicity involves the use of naturally occurring enzymes, and in particular, cholinesterases (ChEs), that are able to bind to or catalyze the hydrolysis of nerve agents. Enzymes have several unique advantages over other approaches. They are catalytically efficient, specific, operate under physiological conditions and rarely cause side effects. The use of enzymes as therapeutic agents is not new to the medical field. Enzymes are used to treat rare genetic disorders (cystic fibrosis, Gaucher disease, severe combined immunodeficiency disease), in wound healing (collagenase), for fibrinolysis (streptokinase, urokinase, tissue plasminogen activator (TPA)) and removal of metabolites in cancer (L-asparaginase).

The use of cholinesterases as “bioscavengers” has been successfully demonstrated in rodents and non-human primates. Administration of sufficient exogenous human BuChE protected mice and rats from multiple lethal-dose organophosphate intoxication (Raveh et al., 1993; Raveh, et al., 1997; Allon et al., 1998). In in vivo studies, endogenous administration of purified ChEs successfully prevented both rodent and primates from poisoning by OP compounds when the nerve agent was dosed up to 5-fold higher than what would normally be lethal (5 LD₅₀) (Raveh et al., 1993; Brandeis et al., 1993; Raveh et al., 1997). Recently, researchers have demonstrated that PON1 also is capable of breaking down organophosphates and protecting test animals from lethal doses of organophosphate pesticides (Costa et al., 1999). Pretreatment of rhesus monkeys with fetal bovine serum-derived acetylcholinesterase (AChE) or horse serum-derived butyrylcholinesterase (BuChE) protected them against a challenge of two to five times the LD₅₀ of pinacolyl methylphosphonofluoridate (soman), a highly toxic organophosphate compound used in chemical warfare (Broomfield et al., 1991; Wolfe et al., 1992). In addition to preventing lethality, the prophylatic treatment prevented behavioral incapacitation after the soman challenge, as measured by probe recognition and equilibrium platform performance tasks.

Purified human BuChE also has a history of use in the treating humans. The literature reports that 134 patients have received partially purified human BuChE for countering the effects of muscle relaxants (succinylcholine and mivacuruium) or for the treatment of organophosphorus pesticide poisoning (Cascio et al., 1998; Table 3 in Duysen et al., 2002). The BuChE injected into these patients was a 5% pure preparation from human plasma and produced by Behringwerke (Germany). The patients recovered with no significant adverse immunological or psychological effects from the BuChE treatment. These studies were performed in Europe and Saudi Arabia, as BuChE has not been approved for human use in the United States.

To make a “bioscavenger” approach practical for human use in a military or civilian setting, the enzyme will need to possess high catalytic activity, good in vivo stability, and a long circulating half-life, particularly if given prophylactically. Unfortunately, most enzymes such as the recombinant AChEs and ChEs have relatively short residence times in circulation, on the order of minutes in mice (Saxena et al., 1998).

Therefore, there remains a need in the art for improved therapeutic agents and methods to combat the toxic effects of OP agents as well as the effects of other compounds, such as pesticides and drugs of abuse.

### SUMMARY OF THE INVENTION

One embodiment of the present invention relates to an isolated PEGylated butyrylcholinesterase protein, comprising at least one polyethylene glycol attached to at least one amino acid of a butyrylcholinesterase protein.

In one embodiment, a polyethylene glycol is attached to at least one lysine or cysteine residue in said butyrylcholinesterase protein. For example, the cysteine residue can include, but is not limited to, Cys66 and Cys571, with respect to SEQ ID NO:1. The lysine residue can include, but is not limited to, K9, K12, K44, K45, K51, K103, K105, K131, K180, K248, K262, K313, K314, K323, K348, K407, K408, K458, K469, K476, K494, and K513, with respect to SEQ ID NO:1.

In another embodiment, a polyethylene glycol is attached to the amino-terminal amino acid of said butyrylcholinesterase protein.

In another embodiment, the polyethylene glycol is attached to at least one added thiol in said butyrylcholinesterase protein. For example, the added thiol can be attached to at least one amino group, to at least one lysine residue, or to the amino-terminal amino acid of the butyrylcholinesterase protein.

In another embodiment, a polyethylene glycol is attached to at least one cysteine residue substituted for at least one amino acid in said butyrylcholinesterase protein. For example, a polyethylene glycol can be attached to at least one cysteine residue substituted for at least one amino acid selected from: asparagine-17, asparagine-57, asparagine-106, asparagine-241, asparagine-256, asparagine-341, asparagine-455, asparagine-481, and asparagine-486 of said butyrylcholinesterase protein, with respect to SEQ ID NO:1. In another aspect, a polyethylene glycol can be attached to at least one cysteine residue substituted for at least one amino acid selected from: E1, D2, D3, I4, I5, I6, A7, T8, K9, N10, G11, K12, R14, G15, M16, N17, L18, and T19, with respect to SEQ ID NO:1. In yet another aspect, a polyethylene glycol can be attached to at least one cysteine residue substituted for at least one amino acid selected from: R520, T523, S524, F525, P527, K528, V529, and any of positions 530 to 574, with respect to SEQ ID NO:1. In yet another aspect, a polyethylene glycol can be attached to at least one cysteine residue substituted for at least one amino acid in the first 7 residues of said butyrylcholinesterase protein or in the last 50 residues of said butyrylcholinesterase protein. In another aspect, a polyethylene glycol is attached to at least one cysteine residue added preceding the first or following the last amino acid of the mature form of said butyrylcholinesterase protein.

In one embodiment, the butyrylcholinesterase protein is truncated after position 530, with respect to SEQ ID NO:1.

In one aspect of any of the above embodiments, the butyrylcholinesterase protein can include at least one mutation that enhances the catalytic activity of the butyrylcholinesterase, wherein the mutation is at an amino acid position selected from: W82, W112, G117, Q119, Y128, E197, S198, A199, W231, L277, L286, L298, V288, E325, A328, F329, V331, W430, H438, and Y440.

In another aspect of any of the above embodiment, the butyrylcholinesterase protein comprises an amino acid sequence that is at least 95% identical to SEQ ID NO:1, wherein said protein catalyzes the hydrolysis of a carboxylester.

In yet another aspect of any of the above-embodiments, the butyrylcholinesterase protein is covalently joined to a non-butyrylcholinesterase protein.

Yet another embodiment of the present invention relates to a fusion protein comprising a butyrylcholinesterase protein covalently joined to a non-butyrylcholinesterase protein. In one aspect, the fusion protein comprises butyrylcholinesterase covalently joined to an immunoglobulin (Ig) domain. In another aspect, the immunoglobulin domain is selected from the group consisting of IgG-Fc, IgG-C_{H} and IgG-C_{L}. In another aspect, the fusion protein is dimeric.

Another embodiment of the present invention relates to an isolated nucleic acid molecule encoding of the above-described proteins and fusion proteins.

Yet another embodiment of the present invention relates to a recombinant nucleic acid molecule comprising any nucleic acid molecule described above, operatively linked to at least one expression control sequence.

Another embodiment of the present invention relates to a recombinant host cell that expresses any recombinant nucleic acid molecule described above. In one aspect, the host cell is selected from: a bacterium, a yeast, a mammalian cell, an insect cell, and a plant cell.

Another embodiment of the present invention relates to a non-human organism that has been genetically modified to express any of the recombinant nucleic acid molecules described above. In one aspect, the non-human organism is selected from a plant and an animal.

Yet another embodiment of the present invention relates to a method to detoxify a carboxylester compound, comprising contacting the compound with any of the PEGylated proteins or fusion proteins described herein.

### DETAILED DESCRIPTION OF THE INVENTION

The present invention generally relates to novel detoxifying enzyme variants and/or conjugates with improved in vivo characteristics as compared to the native (wild-type or unmodified) enzyme, such as an increased circulating half-life and improved potency. This is generally achieved by coupling an enzyme such as butyrylcholinesterase (BuChE) to a polymer or protein, either chemically or genetically (described in detail below). The novel protein adducts of the present invention display apparent molecular weights that are significantly larger than the native protein. This increase in size slows renal clearance and results in a longer acting, potent protein therapeutic.

The first embodiment of the present invention described herein is the chemical coupling of one or more polymers, such as a polyethylene glycol (PEG) moiety, to a detoxifying enzyme such as BuChE. The PEG moiety can be attached to the N-terminal amino acid, a cysteine residue (either native or non-native) or other thiol group, a lysine, or other reactive native or non-native amino acids in the enzyme's primary sequence. A non-native amino acid is defined herein as an amino acid that is not normally located at that position in the protein, an amino acid analog that is not commonly seen in native proteins, or an amino acid or amino acid analog that has been chemically modified to allow conjugation with a polymer such as polyethylene glycol. The specific aspects of this embodiment of the invention are described in detail below.

The second embodiment of the present invention described herein is the production of long acting forms of a detoxifying enzyme such as BuChE that are created through covalent fusion of this protein to a non-butyrylcholinesterase protein. Preferably, BuChE is covalently fused to an immunoglobulin domain such as the Fc (Hinge-C_{H}2—C_{H}3) or complete heavy chain (C_{H}1-Hinge-C_{H}2—C_{H}3) domains of human IgGs, preferably IgG1 and IgG4, or to the constant region of an immunoglobulin light chain, C_{L}. Human IgG1 and IgG4 have long serum half-lives, on the order of 21 days. Fusion of several other proteins, principally extracellular domains of cell surface receptors, to the IgG1 heavy chain domain has resulted in an increased serum half-live for each of these proteins. The specific aspects of this embodiment of the invention are described in detail below. This embodiment of the invention can be combined with the first embodiment of the invention described above, to further enhance the circulating half-life and potency of such agents.

The modified enzymes described herein have the advantage that that they can be dosed less frequently, or at lower doses, than the unmodified parent protein. They are also able to detoxify a wide spectrum of nerve agents or pesticides, making them a “universal” antidote. These compounds also find use as cocaine- or heroin-detoxifying agents for patients that are admitted to hospitals with drug overdoses.

The present invention also includes methods of using any of the detoxifying enzyme variants and/or conjugates to prevent or reduce toxic effects on an individual that result from exposure to a toxic agent, such as organophosphorus (OP) nerve agents, various pesticides and certain drugs of abuse such as cocaine or heroin. Accordingly, the present invention also includes compositions comprising such detoxifying enzyme variants and/or conjugates that can be used in therapeutic or preventative applications.

### Detoxifying Enzyme Variants and Conjugates Useful in the Present Invention

As discussed above, the present invention relates to isolated detoxifying enzyme variants or conjugates with improved bioactivity, and particularly, improved circulating half lives and potency, as compared to the wild-type or native enzymes from which they were derived. Therefore, one embodiment of the present invention relates to an isolated protein, wherein the isolated protein is a detoxifying enzyme (and preferably a cholinesterase, and more preferably butyrylcholinesterase (BuChE)), wherein the enzyme has been modified, as compared to the wild-type or native enzyme, to improve the circulating half-life and/or the potency of the protein, as compared to the wild-type or native enzyme. Such modifications include genetic and post-translational modifications, and the proteins of the invention include any of the variants and/or conjugates described herein (with variants or conjugates having combinations of the modifications being included in the invention).

According to the present invention, a detoxifying enzyme includes any enzyme that acts on a toxic agent (substrate) and catalyzes the conversion (or at least one step of the conversion) of the toxic agent to an agent that is less toxic or non-toxic, particularly with reference to its effect on cells and/or tissues of an organism. Preferred detoxifying enzymes for use in the present invention include, but are not limited to, carboxyl/cholinesterases (CE/ChEs) (e.g., carboxylesterase 1 (hCE1), carboxylesterase 2 (hCE2), acetylcholinesterase (ACHE) and butyrylcholinesterase (BuChE)), phosphoric triester hydrolase enzymes (e.g., organophosphorus hydrolase (OPH)), and paraoxonase/arylestereases (e.g., PON1, PON2 and PON 3). More preferably, detoxifying enzymes for use in the present invention include cholinesterases. Most preferably, cholinesterases useful in the present invention include acetylcholinesterase (AChE) and butyrylcholinesterase (BuChE), with BuChE being most preferred. However, it is to be understood that various enzyme conjugates and modified enzymes described herein using BuChE as an example can be extrapolated to other detoxifying enzymes using the guidance provided herein.

According to the present invention, a “variant” or “homologue” differs from the wild-type (native) protein by one or more minor modifications or mutations to the naturally occurring protein, but maintains the overall basic protein and side chain structure of the naturally occurring form (i.e., such that the variant is identifiable as being related to the wild-type protein). Such changes include, but are not limited to: changes in amino acid side chains; changes amino acids, including deletions (e.g., a truncated version of the protein or peptide) insertions and/or substitutions; changes in stereochemistry of atoms; and/or minor derivatizations, including but not limited to: methylation, farnesylation, geranyl geranylation, glycosylation, carboxymethylation, phosphorylation, acetylation, myristoylation, prenylation, palmitation, and/or amidation. A homologue can have either enhanced, decreased, or substantially similar properties as compared to the naturally occurring protein or peptide. Preferred homologues of a protein are described in detail below. It is noted that variants can include synthetically or recombinantly produced variants.

A conjugate can include any wild-type protein or any variant thereof of the present invention as described herein, wherein the protein has been modified to be conjugated (linked) to another moiety. The moiety can include, but is not limited to, another protein or peptide (as in a fusion or chimeric protein) or another compound, such as a polymer (e.g., polyethylene glycol) as described herein.

Butyrylcholinesterase for Use as an Effective Enzyme-Based Bioscavenger

BuChE is a preferred cholinesterase for human use. BuChE reacts rapidly with all of the highly toxic OPs, offering a broad range of protection from nerve agents, making it a “universal antidote”. In addition, this enzyme is capable of degrading a number of pesticides (Parathion, Malathion) along with several drugs of abuse (cocaine and heroin) (Schwarz et al., 1995; Zhan et al., 2003). Second, BuChE is a human protein and thus should not elicit an immune response, even with repeated administrations. Third, BuChE has proven to be a useful bioscavenger of nerve agents in rodents and in non-human primates. Fourth, researchers have been able to introduce catalytically favorable mutations to enhance reaction rates and catalytic turnover (Saxena et al., 1997). Prior to the present invention, one drawback that has precluded the development of an efficient BuChE-based recombinant bioscavenger has been the short circulatory residence times of the various recombinant preparations. In the animal models, recombinant AChE and BuChE produced in mammalian cells were both eliminated from the bloodstream within a short period of time, on the order of 100 and 60 minutes, respectively (Kronman et al., 1995; Kronman et al., 2000; Chitlaru et al., 1995; Chitlaru et al., 2002; Saxena et al., 1998). The successful application of recombinant ChEs as detoxifying drugs will largely depend on their ability to remain at therapeutic plasma levels for prolonged periods of time (Raveh et al., 1989).

Human BuChE is a 65 kDa glycosylated protein with nine potential Asn-linked carbohydrate chains. The amino acid sequence encoding mature human BuChE is represented herein by SEQ ID NO:1. The nucleotide sequence encoding human BuChE is also known in the art and is represented herein by SEQ ID NO:10 (cds spanning from position 158-1966), which encodes the protein with a signal peptide, represented herein by SEQ ID NO:11. BuChE nucleic acid and amino acid sequences from other species are also known in the art (e.g., see mouse BuChE in GenBank™ Accession No. NM_{—}009738.2 (GI:71896664), or rat BuChE in GenBank™ Accession No. NM_{—}022942.1 (GI:12621109), each of which is incorporated herein by reference in its entirety). When the recombinant form of human BuChE is expressed in Chinese Hamster Ovary (CHO) cells, a mixture of forms is typically recovered (15-40% monomers, 50-55% dimers and 10-30% tetramers). Deletion studies suggest that the aromatic-rich C-terminus (amino acids 530-574) may be responsible for producing the heterogeneous mixture (Blong et al, 1997; Lockridge et al., 1987a; Nicolet et al., 2003). BuChE is most closely related to acetylcholinesterase (AChE; the amino acid sequence of which is represented herein by SEQ ID NO:2) with 53% identical primary sequence. In vivo ACHE terminates the action of the neurotransmitter acetylcholine at postsynaptic membranes and neuromuscular junctions. Although BuChE is found in various tissues (liver, intestine, lung heart, muscle, and brain) its physiological role remains undetermined. This enzyme does have toxicological importance, however, because it is able to hydrolyze ester-containing drugs such as succinylcholine, heroin and cocaine (Sun et al., 2002).

Structural Information

Both ACHE and BuChE have been crystallized and their structures solved. The data for AChE show that the active site catalytic triad (Ser203, Glu334 and His447) is found at the bottom of a 20 Å deep cavity that is lined with 14 aromatic residues. In addition, a peripheral anionic site is located on the outer rim and has been postulated to be the initial substrate binding site (Sussman et al., 1991). The crystal structure for BuChE has only recently become available (Nicolet et al., 2003). As expected, the structure of BuChE is similar to ACHE. The catalytic triad residues in BuChE are Ser198, Glu325, and His438. The main difference between the two proteins is the acyl binding pocket, which is significantly larger for BuChE and is lined with aliphatic residues (leucine and valine) rather than aromatic residues (phenylalanines and tyrosines). This may explain why BuChE is able to hydrolyze a wider variety of cholinesters, while AChE is has a strong preference for acetylcholine.

Mechanism of Action of Cholinesterases and BuChE Variants

Both AChE and BuChE are stoichiometric scavengers in vivo (one molecule of scavenger reacts with one molecule of OP) rather than catalytic scavengers (one molecule of scavenger reacts with many molecules of OP) The inhibition of these enzymes by an organophosphate agent is due to the formation of a stable stoichiometric (1:1) covalent conjugate of the organophosphate with the enzyme's active site serine. Reactivators, such as active site directed nucleophiles (e.g., quaternary oximes), are able to detach the phosphoryl moiety from the hydroxyl group of the active site serine and therefore, are used to treat nerve gas poisoning. In the absence of a reactivator, covalent conjugation of the organophosphate is followed by a second irreversible reaction, known as “aging”, wherein the inhibited ChE is converted into a form that is inactive. This aging process is believed to involve dealkylation of the covalently bound organophosphate group. Through site-directed mutagenesis in the active site of mouse ACHE, researchers have also been able to create a modified enzyme that is more easily reactivated than the wild type enzyme. A mutant enzyme was generated in which the glutamate 202 located amino terminal to the active site serine 203 was converted to a glutamine. The enzyme was resistant to aging, could be readily reactivated by oximes, and was 2-3 times more effective in detoxifying soman and sarin compared to wild type ACHE (Saxena et al., 1997).

Modifications of BuChE that Improve Reactivation

It is one embodiment of the present invention to introduce into BuChE a mutation analogous to the reactivation modification described above for AChE, thus resulting in a more potent molecule in vivo and increasing the likelihood that less protein would be required per treatment, since reactivation with quaternary oximes would now be possible. A long-acting form of BuChE containing an active site mutation for enhanced activity would be a highly improved version of BuChE without the drawbacks of the unmodified parent protein. Specifically, the present invention includes the modification of human BuChE, represented herein by SEQ ID NO:1, by modifying the glutamate-197 located N-terminal to the active site serine-198, by substitution for example, with a glutamine residue. This embodiment of the invention can be combined with any of the other embodiments described elsewhere herein. A similar modification can be made to the corresponding position of BuChE from other species, or in other variants of BuChE, which can be readily identified, for example, by alignment with SEQ ID NO:1.

Additional Genetically Engineered Modifications that Can Enhance Stability of a Long Acting BuChE

Recombinant wild type BuChE is recovered as a mixed population of oligomeric forms (monomers, dimers, and tetramers) due to its hydrophobic C-terminal region that causes aggregation. In order to prepare a stable, homogeneous form of the protein, a truncated version of the protein can be constructed that is fully active and is similar to the protein used to generate crystals for the recent structural studies (Nachon et al., 2002). A portion of the trytophan rich C-terminus which is involved in the non-covalent association of BuChE subunits, can be deleted by placing a stop codon at position 530 (with reference to SEQ ID NO:1). C400 (also with reference to SEQ ID NO:1) can also be genetically mutated to a non-cysteine amino acid such as serine or alanine. The C400S mutation has been shown to not adversely affect the activity of the protein but does aid in its in vitro stability (Nachon et al., 2002). Therefore, these two modifications (A531-574 and C400S) can be introduced into the recombinant long acting BuChE proteins for a more stable protein formulation, if desired. Similar modifications can be made to the corresponding position of BuChE from other species, or in other variants of BuChE.

Additional Mutations that can Increase the Biological Activity of Long Acting BuChEs

The amino acids located near or in the active sites are reasonable candidates for amino acid substitutions to enhance the catalytic activity or enzymatic efficiency of ChEs. Examples of these sties for BuChE (with reference to SEQ ID NO:1) include but are not limited to the catalytic triad (S198, E325 and H438) and amino acids W82, W112, G117, Q119, Y128, A199, W231, L277, L286, L298, V288, A328, F329, V331, W430 and Y440. In addition, amino acids in close proximity to these sites (e.g., from 1 to about 5 amino acids on either side of the reference site) may also be changed to other amino acids to enhance the catalytic efficiency or enzymatic efficiency of the ChE. Several active site mutations that enhance the catalytic activity of ChEs or prevent aging have been reported in the literature. Examples include, but are not limited to, amino acid variants G117H and E197Q, the latter of which is located amino terminal to the active site serine (198) and is analogous to the E202Q mutation described for ACHE, discussed above (Lockridge et al., 1997); Saxena et al., 1997). In addition, more than one active site mutation can be introduced into long acting BuChE variants (PEGylated BuChE or immunofusion constructs described below) by standard genetic engineering approaches.

Covalent Modification of BuChE with Polyethylene Glycol (PEG) to Extend Half-Life

One embodiment of the present invention relates to a detoxifying enzyme, and more preferably, a cholinesterase, and most preferably, a BuChE enzyme, including wild-type BuChE or any variant or truncated form described herein, where the enzyme has been covalently modified with one or more polymers, such as a polyethylene glycol (PEG) moiety, a sugar moiety or a starch moiety. As mentioned above, the polymer moiety can be attached to the N-terminal amino acid of the enzyme, a cysteine residue (either native or non-native) or other thiol group, a lysine, or other reactive native or non-native amino acids in the enzyme's primary sequence.

Covalent modification of proteins with PEG has proven to be a useful method to extend the circulating half-lives of proteins in the body (Abuchowski et al., 1984; Hershfield et al, 1987; Meyers et al., 1991; Keating et al., 1993). Several PEGylated proteins are approved for use in humans or are in human clinical trials (Harris et al., 2003). Covalent attachment of PEG to a protein increases the protein's effective size and reduces its rate of clearance from the body, presumably through interference with protein removal pathways, including kidney glomerular filtration, proteolytic degradation as well as active clearance via specific receptors (Sheffield, 2001; Harris et al., 2001, 2003).

PEGs are commercially available in several sizes and shapes (4-60 kDa, linear and branched), allowing the circulating half-lives of PEG-modified proteins to be tailored for individual indications through the use of different PEGs. PEGylation increases a protein's effective molecular weight more than would be expected based on the molecular weight of the PEG moiety due to the water of hydration associated with the PEG group. For example, attachment of a single 5 kDa PEG to a 36 kDa protein increases the effective molecular weight of the complex to greater than 100 kDa, as measured by size-exclusion chromatography (Fee, 2003). When administered by subcutaneous injection, PEGylated proteins are slowly absorbed from the injection site, thus avoiding the serum “spikes” seen after subcutaneous injection of an unmodified protein. This “controlled release” of the PEGylated protein results in a more constant serum level, thus prolonging or increasing the drug's pharmacologic activity while minimizing the side effects typically seen with fluctuations in the drug concentrations. Other documented in vivo benefits of PEG modification include an increase in protein solubility, enhanced stability (possibly due to protection from proteases) and a decrease in immunogenicity (Katre et al., 1987; Katre, 1990; Keating et al., 1993).

Amine-Reactive PEGs for BuChE PEGylation

The most common route for PEG conjugation of proteins has been to use a PEG with a functional group that reacts with lysines and/or the N-terminal amino acid group. The literature describes more than a dozen such procedures (see reviews by Hooftman et al., 1996; Delgato et al., 1992; and Zalipsky, 1995). Examples of amine-reactive PEGs include, but are not limited to, PEG dichlorotriazine, PEG tresylate, PEG succinimidyl carbonate, PEG benzotriazole carbonate, PEG p-nitrophenyl carbonate, PEG carbonylimidazole, PEG succinimidyl succinate, PEG propionaldehyde, PEG acetaldehyde, and PEG hydroxysuccinimide.

BuChE has 35 lysine residues (Nicolet et al., 2003). Multiple attachments may occur if the protein is exposed to an excess amount of PEGylation reagent. Preferably, the BuChE PEG conjugate would have 1-35 PEGs attached to the protein, more preferred would be 1-10 attachments, and most preferred would be 1-5 attachments. Some preferred lysine residues for PEGylation, with reference to SEQ ID NO:1, include K9, K12, K44, K45, K51, K103, K105, K131, K180, K248, K262, K313, K 314, K323, K348, K407, K408, K458, K469, K476, K494, and K513.

Conditions can be adjusted to limit the number of attachments or the site of attachments. First, the number of attachments can be titrated by varying the molar ratios of the PEG:Protein. Preferred ratios can be determined experimentally. A second method for varying the number of attachments is by modifying the reaction conditions. For example, the coupling can be preferentially directed to the alpha-amine of the N-terminal amino acid of a protein chain by performing the reaction at a pH lower than 7 and preferably below 6.5. Above pH 8, the epsilon-NH3 groups found on the lysines will be most reactive. (Morpurgo and Veronese, 2004). Multiple attachments (5 or less chains) of PEG to an enzyme would be expected to have minimal effects on activity, particularly if the enzyme's substrate is a small molecule and not subject to steric hindrance. A third approach to controlling the number or location of the PEG conjugates is to conduct the PEGylation in the presence of a substrate or reversible inhibitor so that the active site is protected during coupling. A fourth approach to controlling the number of attachments involves using a larger PEG, such as a 10 kDa- to 60 kDA-PEG. For example, when interferon-alpha is modified with a small linear polymer, up to 11 positional isomers are present in the final mixture. When interferon-alpha is modified with a larger 40 kDa branched PEG, only four main positional isomers are present in the mono-PEGylated protein (Monkarsh et al., 1997, Foser et al. 2003, Baillon et al. 2003). A fifth method to control the number of attached PEGs is to use column chromatography procedures (including but not limited to ion exchange, size exclusion or hydrophobic interaction column chromatography) to purify a BuChE conjugate containing the desired number of PEG molecules from a more complex BuChE-PEG mixture.

PEG-BuChE Conjugates Using Thiol-Reactive PEGs

A second method for PEGylating proteins, called site-specific PEGylation, covalently attaches PEG to cysteine residues using thiol-reactive PEGs. A number of highly specific, thiol-reactive PEGs with different reactive groups including, but not limited to, maleimide and vinylsulfone, and different size PEGs (2-60 kDa, linear and branched) are commercially available. The conjugates are hydrolytically stable and the PEGylation reactions can be performed at neutral pH.

BuChE contains 8 native cysteines, six of which form 3 disulfides (C65-C92, C252-C263, C400-C519) and two (C66, C571) that remain free (i.e. cysteine residues not involved in disulfide bonds) (all positions given are relative to SEQ ID NO:1). These free cysteines are reasonable sites for thiol specific PEGylation. Alternatively, by using site-directed mutagenesis, the two free cysteines at positions 66 and 571 can be changed to non-cysteine amino acids, such as serines or alanines, making them unreactive to thio specific PEGylation reagents. Additional free cysteine residues can now be introduced at specific sites in the protein, or inserted between two adjacent amino acids, or added to the N-terminus preceding the first amino acid or added to the C-terminus following last amino acid of the protein. The newly added “free” cysteine serves as the site for the specific attachment of a PEG molecule. The added cysteine preferably is exposed on the protein's surface and accessible for PEGylation. If the chosen site is non-essential, then the PEGylated protein will display wild type (normal) in vitro bioactivity. Preferred sites for the introduction of free cysteines, and in particular, cysteines for PEGylation, are described below.

A free thiol group can also be introduced into the primary amino acid sequence of a protein by chemical modification of an amine group present on the N-terminal amino acid or on a lysine residue. One such example involves treatment of the protein with Traut's reagent. Alternatively the protein can be treated with reagents such as N-succinimidyl S-acetylthioacetate (SATA) or N-succinimidyl S-acetylthioprpionate (SATP) that introduces a protected sulfydryl which can be deprotected prior to exposure to a thiol reactive PEG. Alternatively, a “free” cysteine can be introduced by deleting or mutating a native cysteine (that normally forms a disulfide bond) to another amino acid such as a serine or alanine so that an odd number of cysteines are present in the protein's primary sequence.

Free thiol groups can also be introduced by chemical conjugation of a peptide to a protein where the peptide contains a free cysteine group or a cysteine group modified with a reversible thiol blocking agent.

Selecting Sites in BuChE for Cysteine Mutagenesis (or Thiol Modification) and PEGylation

Glycosylation sites are preferred sites for attaching PEG molecules to proteins because (1) these sites are surface exposed, (2) the natural protein can typically tolerate bulky sugar groups at this position, and (3) glycosylation sites are often located away from regions critical for biological activity. In the case of BuChE, glycosylation is not required for in vitro activity (Nachon et al., 2002). The human BuChE sequence contains nine potential N-linked glycosylation sites (N17, N57, N106, N241, N256, N341, N455, N481, and N486). All are preferred targets for site-specific cysteine substitution or thiol modification and subsequent PEGylation according to the present invention, particularly since this is an enzyme with a small substrate and since all of the glycosylation sites are a reasonable distance from the active site of the enzyme. Again, all positions are given with regard to SEQ ID NO:1.

The N-terminal and C-terminal amino acids of BuChE are not required for biological activity based on deletion studies, and thus, are also preferred locations for site-specific PEGylation (Blong et al., 1997). Cysteine substitutions (or thiol modification) or insertions introduced in the first about 7 amino acids (i.e., E1, D2, D3, I4, I5, I6, or A7) or within the last about 50 amino acids (i.e., any amino acid including and between about F525 and L574) would be expected to have only minimal effects on activity. Preferred sites for cysteine substitutions (or thiol modification) in the N-terminal portion of BuChE include: E1, D2, D3, I4, I5, I6, A7, T8, K9, N10, G11, K12, R14, G15, M16, N17, L18, and T19. In addition to any of positions 531-574, other preferred sites for cysteine substitution (or thiol modification) in the C-terminal region of BuChE include, but are not limited to, R520, T523, S524, F525, P527, K528, and V529. All positions are given with regard to SEQ ID NO:1.

ChE Fusion Proteins

The present invention also includes the use of recombinant DNA technology to covalently fuse the protein of interest (e.g., a detoxifying enzyme, and more preferably, a cholinesterase, and most preferably, a BuChE enzyme, including wild-type BuChE or any variant or truncated form described herein) to a second protein that naturally has a long circulating half-life. Examples of proteins that naturally have long half-lives include, but are not limited to, human IgGs, albumin, transferrin and transferrin receptor proteins. One protein that has a long circulating half-life and which has been used to create numerous fusion proteins is human IgG1. IgG1 is the most common immunoglobulin in serum (70% of total IgG) and has a serum half-life of 21 days (Capon et al., 1989; Roitt et al., 1989). Although less abundant (5% of total IgG), IgG4 also has a long circulating half-life of 21 days. IgG4 displays reduced ability to activate complement and bind Fc receptors relative to IgG1, which may make it a preferred choice for use as a human therapeutic (Roitt et al., 1989).

Human IgGs have a multi-domain structure, comprising two light chains disulfide-bonded to two heavy chains. Each light chain and each heavy chain contains a variable region joined to a constant region. The variable regions are located at the N-terminal ends of the light and heavy chains. The heavy chain constant region is further divided into C_{H}1, Hinge, C_{H}2 and C_{H}3 domains. The C_{H}1, C_{H}2 and C_{H}3 domains are discreet domains that fold into a characteristic structure. The Hinge region is a region of considerable flexibility. The various heavy chain domains are encoded by different exons in the IgG genes (Ellison et al., 1981; 1982). Proteins also can be fused to the constant region of immunoglobulin light chains, preferably at the N-terminus of the light chain constant region.

Proteins have been fused to the heavy chain constant region of IgGs at the junction of the variable and constant regions (thus containing the C_{H}1-Hinge-C_{H}2—C_{H}3 domains—referred to as C_{H} fusions) and at the junction of the C_{H}1 and Hinge domains (thus containing the Hinge-C_{H}2—C_{H}3 domains—referred to as Fc fusions). The IgG-C_{H} fusions create larger proteins that are expected to have longer circulating half-lives. IgG1 and IgG4 heavy chains normally form disulfide-linked dimers through cystine bonds located in the Hinge region. Since the Hinge region will be present in all of the proposed fusion proteins, the fusion proteins are usually dimeric.

The proteins may be fused with or without a linker peptide present. Preferred linkers are 1-50 amino acids in length, more preferred is 1-15, and most preferred is 1-10 amino acids. It is also preferred that the linkers are flexible and do not significantly interfere with the therapeutic protein's activity. Preferred linkers contain a mixture of amino acids, including glycine, serine, alanine, and threonine residues, or any combination of these residues. Methods for constructing fusion proteins without a peptide linker sequence are provided in PCT Publication No. Publication No. WO 01/03737 A1.

### Nucleic Acid Molecules

The present invention also includes isolated nucleic acid molecules comprising, consisting essentially of, or consisting of a nucleic acid sequence encoding any of the above-described detoxifying enzyme variants or conjugates (in the case of fusion proteins) described above. The present invention further includes recombinant nucleic acid molecules comprising such nucleic acid sequences, as well as recombinant host cells that express such molecules.

Compositions and Methods for Using the Detoxifying Enzyme Variants and Conjugates of the Present Invention

The present invention also includes compositions and methods for using the detoxifying enzyme variants and conjugates of the invention. In particular, the present invention includes the use of any of the detoxifying enzyme variants and conjugates described herein to prevent or reduce the effects resulting from exposure of an individual to a toxic agent that can be detoxified by a protein described herein, including, but not limited to, organophosphorus (OP) nerve agents, various pesticides and certain drugs of abuse such as cocaine or heroin. Therefore, the invention encompasses methods of administering to an individual to be protected from such toxic compounds (by prevention or treatment after exposure) one or more detoxifying enzyme variants and conjugates of the invention or a composition comprising such detoxifying enzyme variants and conjugates.

A composition includes any of the detoxifying enzyme variants and conjugates of the invention, or nucleic acid molecules encoding such variants or conjugates, and the composition can further include, for example, a pharmaceutically acceptable carrier, which includes pharmaceutically acceptable excipients and/or delivery vehicles, for delivering the protein or nucleic acid molecule to an individual. As used herein, a pharmaceutically acceptable carrier refers to any substance suitable for delivering a composition useful in the method of the present invention to a suitable in vivo or ex vivo site.

### General Definitions and Description Related to Embodiments of the Invention

According to the present invention, an isolated protein is a protein or a fragment thereof (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. An isolated peptide can be produced synthetically (e.g., chemically, such as by peptide synthesis) or recombinantly.

Reference to a particular protein from a specific organism or to a particular protein being derived from a specific organism, such as a “human butyrylcholinesterase” or a “butyrylcholinesterase derived from human”, by way of example, refers to a butyrylcholinesterase (including a homologue of the naturally occurring butyrylcholinesterase) from a human or a butyrylcholinesterase that has been otherwise produced from the knowledge of the structure (e.g., sequence) of a naturally occurring butyrylcholinesterase from human. In other words, a human butyrylcholinesterase includes any butyrylcholinesterase that has the structure and function of a naturally occurring butyrylcholinesterase from human or that has a structure and function that is sufficiently similar to a human butyrylcholinesterase such that the butyrylcholinesterase is a biologically active (i.e., has biological activity) homologue (variant) of a naturally occurring butyrylcholinesterase from human. As such, a human butyrylcholinesterase can include purified, partially purified, recombinant, mutated/modified and synthetic proteins. The term “variant” or “homologue” has been defined previously herein.

According to the present invention, the terms “modification” and “mutation” can be used interchangeably, particularly with regard to the modifications/mutations to the primary amino acid sequences of a protein or peptide (or nucleic acid sequences) described herein. The term “modification” can also be used to describe post-translational modifications to a protein or peptide including, but not limited to, methylation, farnesylation, carboxymethylation, geranyl geranylation, glycosylation, phosphorylation, acetylation, myristoylation, prenylation, palmitation, amidation and/or PEGylation. Modifications can also include, for example, complexing a protein or peptide with another compound. Such modifications can be considered to be mutations, for example, if the modification is different than the post-translational modification that occurs in the natural, wild-type protein or peptide.

Conservative substitutions referenced herein typically include substitutions within the following groups: glycine and alanine; valine, isoleucine and leucine; aspartic acid, glutamic acid, asparagine, and glutamine; serine and threonine; lysine and arginine; and phenylalanine and tyrosine. Substitutions may also be made on the basis of conserved hydrophobicity or hydrophilicity (Kyte and Doolittle, J. Mol. Biol. 157:105 (1982)), or on the basis of the ability to assume similar polypeptide secondary structure (Chou and Fasman, Adv. Enzymol. 47: 45 (1978)).

Variants can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications or mutations in protein homologues, as compared to the wild-type protein, either increase, decrease, or do not substantially change, the basic biological activity of the homologue as compared to the naturally occurring (wild-type) protein. Preferably, the modifications increase or enhance the activity of the protein, for example by increasing or enhancing the ability of the protein to detoxify a substrate through enzymatic conversion (e.g., hydrolysis) of the toxic substrate to another compound, or by increasing the circulating half-life or potency (e.g., less of the protein is needed to achieve the same result, as compared to the native protein) of the protein. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Biological activities of a detoxifying enzyme, such as butyrylcholinesterase, include binding to a substrate and catalyzing the hydrolysis of a toxic compound (e.g., a carboxylester).

Modifications of a protein, such as in a variant, may result in proteins having the same biological activity as the naturally occurring protein, or in proteins having decreased or increased biological activity as compared to the naturally occurring protein. It is noted that general reference to a homologue having the biological activity of the wild-type protein does not necessarily mean that the homologue has identical biological activity as the wild-type protein, particularly with regard to the level of biological activity. Rather, a variant can perform the same biological activity as the wild-type protein, but at a reduced or increased level of activity as compared to the wild-type protein. Methods to evaluate the biological activity of the variants of the invention can include assays that are useful for evaluating activity of the native protein and are also described herein.

In one aspect of the invention, a protein encompassed by the present invention, including a variant of a particular protein described herein, comprises an amino acid sequence that includes at least about 100 consecutive amino acids of the amino acid sequence from the reference protein, wherein the amino acid sequence of the homologue has a biological activity of the protein as described herein. In a further aspect, the amino acid sequence of the protein is comprises at least about 150 consecutive amino acids, and more preferably at least about 200 consecutive amino acids, and more preferably at least about 250 consecutive amino acids, and more preferably at least about 300 consecutive amino acids, and more preferably at least about 350 consecutive amino acids, and more preferably at least about 400 consecutive amino acids, and more preferably at least about 500 consecutive amino acids of the amino acid sequence of the reference protein.

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

Typically, a variant of a reference protein, such as any of the detoxifying enzymes described herein, has an amino acid sequence that is at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical, and more preferably at least about 90% identical, and more preferably at least about 95% identical, and more preferably at least about 96% identical, and more preferably at least about 97% identical, and more preferably at least about 98% identical, and more preferably at least about 99% identical (or any percentage between 60% and 99%, in whole single percentage increments) to the amino acid sequence of the reference protein (e.g., to a native butyrylcholinesterase). The homologue preferably has a biological activity of the protein from which it is derived or related (i.e., the protein having the reference amino acid sequence).

As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, “Blast 2 sequences—a new tool for comparing protein and nucleotide sequences”, FEMS Microbiol Lett. 174:247 (1999), incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:
Reward for match = 1
Penalty for mismatch = −2
Open gap (5) and extension gap (2) penalties
gap x_dropoff (50) expect (10) word size (11) filter (on)
For blastp, using 0 BLOSUM62 matrix:
Open gap (11) and extension gap (1) penalties
gap x_dropoff (50) expect (10) word size (3) filter (on).

In another embodiment of the invention, an amino acid sequence having the biological activity of a protein described herein (e.g., a butyrylcholinesterase) includes an amino acid sequence that is sufficiently similar to the naturally occurring protein or polypeptide that is specifically described herein that a nucleic acid sequence encoding the amino acid sequence is capable of hybridizing under moderate, high, or very high stringency conditions (described below) to (i.e., with) a nucleic acid molecule encoding the naturally occurring protein or polypeptide (i.e., to the complement of the nucleic acid strand encoding the naturally occurring protein or polypeptide). Preferably, an amino acid sequence having the biological activity of a protein described herein is encoded by a nucleic acid sequence that hybridizes under moderate, high or very high stringency conditions to the complement of a nucleic acid sequence that encodes any of the amino acid sequences described herein. Methods to deduce a complementary sequence are known to those skilled in the art. It should be noted that since amino acid sequencing and nucleic acid sequencing technologies are not entirely error-free, the sequences presented herein, at best, represent apparent sequences of the proteins encompassed by the present invention.

As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press (1989). Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., Anal. Biochem. 138, 267 (1984); Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

The present invention also includes a fusion protein that includes any protein or any homologue or fragment thereof of the present invention attached to one or more fusion segments. Suitable fusion segments for use with the present invention include, but are not limited to, segments that can: enhance a protein's stability; provide other desirable biological activity; and/or assist with the purification of the protein (e.g., by affinity chromatography). Particularly preferred fusion segments (partners) are described previously herein. A suitable fusion segment can be a domain of any size that has the desired function (e.g., imparts increased stability, solubility, biological activity; and/or simplifies purification of a protein). Fusion segments can be joined to amino and/or carboxyl termini of the protein and can be susceptible to cleavage in order to enable straight-forward recovery of the desired protein. Fusion proteins are preferably produced by culturing a recombinant cell transfected with a fusion nucleic acid molecule that encodes a protein including the fusion segment attached to either the carboxyl and/or amino terminal end of the protein of the invention as discussed above.

In one embodiment of the present invention, any of the amino acid sequences described herein, as well as homologues of such sequences, can be produced with from at least one, and up to about 20, additional heterologous amino acids flanking each of the C- and/or N-terminal end of the given amino acid sequence. The resulting protein or polypeptide can be referred to as “consisting essentially of” a given amino acid sequence. According to the present invention, the heterologous amino acids are a sequence of amino acids that are not naturally found (i.e., not found in nature, in vivo) flanking the given amino acid sequence or which would not be encoded by the nucleotides that flank the naturally occurring nucleic acid sequence encoding the given amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a given amino acid sequence that can be flanked by from at least one, and up to as many as about 60, additional heterologous nucleotides at each of the 5′ and/or the 3′ end of the nucleic acid sequence encoding the given amino acid sequence. The heterologous nucleotides are not naturally found (i.e., not found in nature, in vivo) flanking the nucleic acid sequence encoding the given amino acid sequence as it occurs in the natural gene.

The minimum size of a protein or domain and/or a homologue or fragment thereof of the present invention is, in one aspect, a size sufficient to have the requisite biological activity, or sufficient to serve as an antigen for the generation of an antibody or as a target in an in vitro assay. In one embodiment, a protein of the present invention is at least about 8 amino acids in length (e.g., suitable for an antibody epitope or as a detectable peptide in an assay), or at least about 25 amino acids in length, or at least about 50 amino acids in length, or at least about 100 amino acids in length, or at least about 150 amino acids in length, or at least about 200 amino acids in length, or at least about 250 amino acids in length, or at least about 300 amino acids in length, or at least about 350 amino acids in length, or at least about 400 amino acids in length, or at least about 450 amino acids in length, or at least about 500 amino acids in length, and so on, in any length between 8 amino acids and up to the full length of a protein or domain of the invention or longer, in whole integers (e.g., 8, 9, 10, . . . 25, 26, . . . 500, 501, . . . ). There is no limit, other than a practical limit, on the maximum size of such a protein in that the protein can include a portion of the protein, domain, or biologically active or useful fragment thereof, or a full-length protein or domain, plus additional sequence (e.g., a fusion protein sequence), if desired.

Another embodiment of the present invention relates to isolated nucleic acid molecules comprising, consisting essentially of, or consisting of nucleic acid sequences that encode any of the proteins described herein, including a homologue or fragment of any of such proteins, as well as nucleic acid sequences that are fully complementary thereto. In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or a domain of a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., retain, improve or decrease activity of the protein). Protein homologues (e.g., proteins encoded by nucleic acid homologues) have been discussed in detail above.

A nucleic acid molecule homologue can be produced using a number of methods known to those skilled in the art (see, for example, Sambrook et al., Molecular Cloning: A Laboratory Manual, Cold Spring Harbor Labs Press (1989)). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The minimum size of a nucleic acid molecule of the present invention is a size sufficient to form a probe or oligonucleotide primer that is capable of forming a stable hybrid (e.g., under moderate, high or very high stringency conditions) with the complementary sequence of a nucleic acid molecule of the present invention, or of a size sufficient to encode an amino acid sequence having a biological activity of a protein according to the present invention. As such, the size of the nucleic acid molecule encoding such a protein can be dependent on the nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a sequence sufficient to encode a biologically active fragment of a protein or the full-length protein.

Another embodiment of the present invention includes a recombinant nucleic acid molecule comprising a recombinant vector and a nucleic acid sequence encoding any of the proteins described herein. According to the present invention, a recombinant vector is an engineered (i.e., artificially produced) nucleic acid molecule that is used as a tool for manipulating a nucleic acid sequence of choice and for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid molecules of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant organism. The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector that enable the transcription and translation of the nucleic acid sequence within the recombinant host cell.

Typically, a recombinant nucleic acid molecule includes at least one nucleic acid molecule of the present invention operatively linked to one or more expression control sequences. As used herein, the phrase “recombinant molecule” or “recombinant nucleic acid molecule” primarily refers to a nucleic acid molecule or nucleic acid sequence operatively linked to a expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence (e.g., a transcription control sequence and/or a translation control sequence) in a manner such that the molecule can be expressed when transfected (i.e., transformed, transduced, transfected, conjugated or conduced) into a host cell. Transcription control sequences are sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those that are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

One or more recombinant molecules of the present invention can be used to produce an encoded product (e.g., butyrylcholinesterase variant) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cell that can be transfected. Host cells can be either untransfected cells or cells that are already transfected with at least one other recombinant nucleic acid molecule.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast, or into plant cells. In microbial and plant systems, the term “transformation” is used to describe an inherited change due to the acquisition of exogenous nucleic acids by the microorganism or plant and is essentially synonymous with the term “transfection.” However, in animal cells, transformation has acquired a second meaning which can refer to changes in the growth properties of cells in culture after they become cancerous, for example. Therefore, to avoid confusion, the term “transfection” is preferably used with regard to the introduction of exogenous nucleic acids into animal cells, and the term “transfection” will be used herein to generally encompass transfection of animal cells, and transformation of microbial cells or plant cells, to the extent that the terms pertain to the introduction of exogenous nucleic acids into a cell. Therefore, transfection techniques include, but are not limited to, transformation, particle bombardment, diffusion, active transport, bath sonication, electroporation, microinjection, lipofection, adsorption, infection and protoplast fusion.

Methods for the genetic engineering of plants are also well known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10:1 (1991). Descriptions of Agrobacterium vector systems and methods for Agrobacterium-mediated gene transfer are provided by numerous references, including Gruber et al., supra, Miki et al., supra, Moloney et al., Plant Cell Reports 8:238 (1989), and U.S. Pat. Nos. 4,940,838 and 5,464,763.

Another generally applicable method of plant transformation is microprojectile-mediated transformation wherein DNA is carried on the surface of microprojectiles. The expression vector is introduced into plant tissues with a biolistic device that accelerates the microprojectiles to speeds sufficient to penetrate plant cell walls and membranes. Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

According to the present invention, a “genetically modified” or “transgenic” animal is a non-human animal that has a genome which is modified (i.e., mutated or changed) from its normal (i.e., wild-type or naturally occurring) form such that the desired result is achieved (e.g., expression of a recombinant nucleic acid molecule of the invention). It is noted that the actual technical step of genetic modification may have occurred in an ancestor (e.g., parent, grandparent, etc.) of the animal, and the resulting genetic modification may therefore exist in a subject animal as a result of breeding and inheritance of the genetic trait. Therefore, reference to genetic modification can include genetic modification that is inherited from an ancestor. Genetic modification of an animal is typically accomplished using molecular genetic and cellular techniques, including manipulation of embryonic cells and DNA. Such techniques are generally disclosed for mice, for example, in “Manipulating the Mouse Embryo” (Hogan et al., Cold Spring Harbor Laboratory Press, 1994, incorporated herein by reference in its entirety).

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts.

Enzyme variants and conjugates of the present invention are preferably administered in a composition. Compositions can include an enzyme variant or conjugate of the invention and any other suitable pharmaceutically acceptable carrier, as well as, in some aspects, additional components that may be useful in the treatment of a condition (e.g., exposure to a nerve agent, a pesticide, or drug addiction). According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably any site where the enzyme variant or conjugate will provide a detectable effect as compared to in the absence of the enzyme variant or conjugate. Preferred pharmaceutically acceptable carriers are capable of maintaining the enzyme variant or conjugate of the present invention in a form that, upon arrival of the enzyme variant or conjugate at a cell or tissue target, the enzyme variant or conjugate is capable of interacting with its target.

Suitable excipients of the present invention include excipients or formularies that transport or help transport, but do not specifically target a composition to a cell or area (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

One type of pharmaceutically acceptable carrier includes a controlled release formulation that is capable of slowly releasing a composition of the present invention in an individual. As used herein, a controlled release formulation comprises an enzyme variant or conjugate of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to an individual, form a solid or a gel in situ. Preferred carriers are also biodegradable (i.e., bioerodible). In the event that an enzyme variant or conjugate of the invention is administered as a recombinant nucleic acid molecule encoding the variant or conjugate, suitable carriers include, but are not limited to liposomes, viral vectors or other carriers, including ribozymes, gold particles, poly-L-lysine/DNA-molecular conjugates, and artificial chromosomes. Natural lipid-containing carriers include cells and cellular membranes. Artificial lipid-containing carriers include liposomes and micelles.

A carrier of the present invention can be modified to target to a particular site in an individual, thereby targeting and making use of an enzyme variant or conjugate of the present invention at that site. A pharmaceutically acceptable carrier which is capable of targeting can also be referred to herein as a “delivery vehicle” or “targeting carrier”. Suitable modifications include manipulating the chemical formula of the lipid portion of the delivery vehicle and/or introducing into the vehicle a targeting agent capable of specifically targeting a delivery vehicle to a preferred site or target site, for example, a preferred tissue type. A “target site” refers to a site in an individual to which one desires to deliver a composition. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Another type of delivery vehicle, when the enzyme variant or conjugate is administered as a nucleic acid encoding the variant or conjugate, comprises a viral vector. A viral vector includes an isolated nucleic acid molecule, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

According to the present invention, an effective administration protocol (i.e., administering a therapeutic composition in an effective manner) comprises suitable dose parameters and modes of administration that result in the desired effect in the patient (e.g., detoxification of a particular compound so that the effect of the compound is eliminated or reduced). Effective dose parameters can be determined using methods standard in the art for enzyme administration. One can also determine the dose parameters using the guidance provided herein. Such methods include, for example, determination of survival rates, side effects (i.e., toxicity) and progression or regression of disease.

In accordance with the present invention, a suitable single dose size is a dose that results in the desired therapeutic effect in the individual, depending on the enzyme variant or conjugate that is administered, or in the amelioration of at least one symptom of a condition in the individual, when administered one or more times over a suitable time period. One of skill in the art can readily determine appropriate single dose sizes for a given individual based on the size of a patient and the route of administration.

In one aspect of the invention an appropriate single dose of an enzyme variant or conjugate of the present invention is at least about 0.01 g per kg of the individual to which the enzyme variant or conjugate is administered, and in other aspects, at least about 0.1 μg/kg, at least about 0.2 μg/kg, at least about 0.5 μg/kg, at least about 11 g/kg, at least about 5 μg/kg, at least about 10 μg/kg, at least about 25 μg/kg, at least about 50 μg/kg, at least about 75 μg/kg, at least about 100 μg/kg, at least about 200 μg/kg, at least about 300 μg/kg, at least about 400 μg/kg, at least about 500 μg/kg, at least about 750 μg/kg, at least about 1 mg/kg, or at least about 5 mg/kg.

As discussed above, a enzyme variant or conjugate or composition comprising the enzyme variant or conjugate of the present invention is administered to an individual in a manner effective to deliver the composition to a cell, a tissue, and/or systemically to the individual, whereby the desired result is achieved as a result of the administration of the composition. Suitable administration protocols include any in vivo or ex vivo administration protocol. The preferred routes of administration will be apparent to those of skill in the art. For proteins or nucleic acid molecules, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. Routes useful for deliver to mucosal tissues include, bronchial, intradermal, intramuscular, intranasal, other inhalatory, rectal, subcutaneous, topical, transdermal, vaginal and urethral routes. Combinations of routes of delivery can be used and in some instances, may enhance the therapeutic effects of the composition. Particularly preferred routes of delivery include subcutaneous and intravenous delivery.

In the method of the present invention, enzyme variants or conjugates or compositions comprising such enzyme variants or conjugates can be administered to any animal and preferably, to any member of the Vertebrate class, Mammalia, including, without limitation, primates, rodents, livestock and domestic pets. Livestock include mammals to be consumed or that produce useful products (e.g., sheep for wool production). Preferred mammals to protect include humans, dogs, cats, mice, rats, sheep, cattle, horses and pigs, with humans being particularly preferred.

The following examples are provided for the purpose of illustration and are not intended to limit the scope of the present invention.

### EXAMPLES

### Example 1

### In vitro Spectrophotometric Assay for BuChE Proteins

The kinetic constants (Kₘ and K_{cat}) of the BuChE protein variants can be determined for the substrates butyrylthiocholine and acetylthiocholine (Sigma) using a variation of the Ellman method as described previously (Platteborze and Broomfield, 2000; Ellman et al., 1961). Enzymatic hydrolysis of the substrate generates free thiocholine which reacts with Ellman's reagent (DTNB) to produce a yellow chromophore. The reactions are set up in microtiter plates and monitored at 412 nm. Commercially available equine butyrylcholinesterase can serve as a positive control for the assay along with human recombinant wild type butyrylcholinesterase.

### Example 2

### Cloning, Expression, and Mutagenesis of BuChE

Recombinant expression of BuChE has been reported in E. coli (Masson, 1990), microinjected, Xenopus laevis oocytes (Soreq et al., 1989), insect cell lines in vitro and larvae in vivo (Platteborze and Broomfield, 2000), in silkworms (Wei et al., 2000) and in mammalian COS cells (Platteborze and Broomfield, 2000), CHO cells (Masson et al., 1993; Lockridge et al. 1997) and in transgenic goats (Nexia, Inc.).

A cDNA for Human BuChE was amplified by PCR from human liver, fetal human liver, and human pituitary cDNA libraries (QUICKclone™ cDNA libraries, Clontech). Primers BB1136 and BB1137 (see Table I for list of oligonucleotide primer sequences) were used in the PCR with the cDNA libraries as templates, producing fragments ˜1.6 kbp in length (termed fragment 1136×1137), which were gel-purified. The 1136×1137 fragments were further amplified in two ways. In one method, the fragments were further amplified using primers BB1076, BB1134, BB1135, and BB1141, producing a partial gene fragment (fragment 1076×1141) 875 bp in length and containing translational initiation sequences at the 5′ end of the gene. For the other method, fragments were reamplified with BB1136 and BB1137, producing fragments 1136×1137-2°. The 1076×1141 fragments were digested with NdeI and XbaI (the XbaI site is present in the cDNA at codons 253-254 of the BuChE amino acid sequence), cloned into similarly digested pUC19, and sequenced. The 1135×1136-2° fragments were digested with EcoRI (the EcoRI site is present in the cDNA at codons 30-32 of the BuChE amino acid sequence) and KpnI, and cloned into similarly digested pUC19, and sequenced, or they were digested with XbaI and KpnI, and cloned into similarly digested pcDNA3.1 (Invitrogen), and sequenced.

TABLE I
Oligonucleotide Primers

| | |
|---|---|
| Name of Primer | Sequence |

| | |
|---|---|
| BB1076 (SEQ ID NO: 3) | 5′CGCCATATGGGATCCATCTTGGAGGATGATTAAATG |
| BB1134 (SEQ ID NO: 4) | 5′TTGGAGGATGATTAAATGGAAGATGACATCATT |
| BB1135 (SEQ ID NO: 5) | 5′ATGGAAGATGACATCATTATTGCAACAAAGAATGGTAAAG |
| BB1136 (SEQ ID NO: 6) | 5′TGCAACAAAGAATGGTAAAGTCCGTGGGATGAACTTGAC |
| BB1137 (SEQ ID NO: 7) | 5′CGGGGTACCTTACAAGACTTTTGGAAAAAATGATGTCCA |
| BB1141 (SEQ ID NO: 8) | 5′CAACAAATGCTTCATTCAGAAG |

A comparison of sequences of clones revealed a number of changes from the expected sequence, such as nucleotide insertions and deletions, as well as codons for different amino acids. In fact, none of the eleven clones contained the expected sequence. However, several clones contained stretches of correct sequence flanked by unique restriction sites. Thus, restriction fragments isolated from different clones can be ligated to construct a complete gene for BuChE.

Incorporated into the oligonucleotide primers used in the PCRs are sequences important for efficient translational initiation, as well as restriction sites for cloning into useful vectors for expression in E. coli. For example, the BuChE gene can be inserted into the T7 promoter vector, pET21a+ (Novagen) for expression in the E. coli strain BL21 (DE3), which contains the gene for the T7 RNA polymerase. The BuChE gene can also be inserted into either tetracycline-resistant or kanamycin-resistant derivatives of the T5 promoter expression vector pQE60 (Qiagen) for expression in a number of E. coli strains, such as W3110. Finally, the BuChE gene can be inserted into an appropriately modified tetracycline-resistant derivative of the Tac promoter expression vector pCYB1 (New England Biolabs) for expression in E. coli.

Specific substitutions can be introduced into the BuChE sequence using PCR mutagenesis as described by Higuchi (1990) or Scharf (1990). Preferred sites for introducing a free cysteine residue include glycosylation sites and the N- and C-termini that are removed from the active site. Examples include but are not limited to −1 C (an addition cysteine residue added to the N-terminus); E1C; N17C, N57C, N106C, N241C, N256C, N341C, N455C, N481C, and N486C (all of which are proposed glycosylation sites); and L574C which is the C-terminus of the full length protein, or L530C, which is the C-terminus of a truncated version of BuChE that terminates at amino acid 530. A cysteine residue also can be introduced following the C-terminal amino acid of full length or other truncated BuCHE proteins. In addition to the sites listed in this example, any amino acid present on the surface of the protein can be changed to a cysteine residue. These can be identified from the crystal structure (Nicolet et al., 2003). Preferred sites can also be identified by cysteine scanning mutagenesis of the protein. Particularly preferred sites have been described in detail above.

Intracellularly-expressed BuChE and the BuChE cysteine muteins if recovered in an insoluble form, can be renatured to a fully active conformation following refolding protocols as described previously for BuChE and AChE (Masson, 1990; Fischer et al., 1995) or by using the methods for folding cysteine muteins as described in Rosendahl et al., 2001. The proteins can be purified using previously reported protocols for purifying BuChE that include anion exchange chromatography and affinity chromatography using procainamide-Sepharose column (Masson, 1990; Fischer et al., 1995). If the expressed BuChE and the BuChE cysteine muteins are recovered in a soluble form, but not in a native conformation, the BuChE in the cell free extracts can be refolded before purification or after purification.

Recombinant BuChE and the BuChE cysteine muteins also can be expressed as intracellular or secreted proteins in eukaryotic cells such as yeast, insect cells or mammalian cells. Vectors for expressing the proteins and methods for performing such experiments are described in catalogues from various commercial supply companies such as Invitrogen, Inc., Stratagene, Inc. and CloneTech, Inc. BuChE variants can also be produced in transgenic animals (Goldman, 2003; Baldassarre et al., 2004).

### Example 3

### Cysteine PEGylation Optimization and In vitro Evaluation of PEG-Cys-BuChE Candidates

BuChE cysteine muteins that retain in vitro activity are good candidates for screening studies using a cysteine-reactive 20 kDa PEG-maleimide. The cysteine muteins must be partially reduced with dithiothreitol (DTT), Tris (2-carboxyethyl) phosphine-HCl (TCEP) or other disulfide disrupting agent, in order to expose the free cysteine for PEGylation and allow the PEGylation reaction to proceed efficiently. Typically a 5-fold molar excess of DTT for 30 min is sufficient. Excess DTT can be removed by dialysis. The reduced protein is reacted with various concentrations of PEG-maleimide to determine the optimum ratio. PEGylation of the protein can be monitored by a molecular weight shift using SDS-PAGE. The lowest amount of PEG that gives significant quantities of mono-PEGylated product is considered optimum (80% conversion is considered good). The PEGylated protein is purified from the unPEGylated protein by column chromatography, including but not limited to hydrophobic interaction, ion exchange chromatography and size exclusion chromatography. Concentrations of purified PEGylated muteins can be measured using the Bradford protein assay since the PEG does not interfere with dye binding to the protein. The activity of the purified PEGylated proteins can be determined using the spectrophotometric assay described in Example 1.

### Example 4

### Amine PEGylation of BuChE

Wild type BuChE or BuChE variants (truncated and/or with active site mutations) can also be PEGylated using amine reactive PEG reagents. Because the water hydroxyl anion of the aqueous buffer competes with the primary amines, an excess of active PEG is usually needed, on the order of 2× to 100× depending on the protein's reactivity. The predominant site(s) of PEGylation can be controlled based on the pH of the buffer. Generally, at pH values above 8.0, the epsilon-NH3 groups of lysines react first whereas at approximated pH 5-7, the alpha-NH2 group of the N-terminal amino acid is the most reactive.

For N-terminal PEGylation, BuChE is diluted into a buffer that has sufficient capacity to maintain the pH of the reaction between 5-7. Buffers containing primary amines such as Tris should be avoided. The protein's concentration can be on the order of 0.01 mg-50 mg/ml. PEG is added on the order of 2-100-fold excess, preferably 2-10-fold excess. The reaction is allow to sit overnight at 4° C. or until the reaction is considered complete. The PEGylated protein is separated from the non-PEGylated protein and the PEG reagents by column chromatography procedures, including but not limited to ion exchange, hydrophobic interaction, or size exclusion chromatography. Chromatography can also be used to separate the PEGylated isoforms of the proteins that vary by the location and/or the number of PEGs attached. The proteins can be visualized by UV absorbance at 280 nm whereas the PEG molecules can be identified by iodine assay (Sims et al., 1980).

For non-specific amine PEGylation the above reaction is performed at a pH greater than 7, and preferably greater than 8. The number of attachments desired in the final product can be controlled by the amount of excess PEG reagent added and the time that the reaction is allowed to proceed.

### Example 5

### Construction, Expression and Purification of BuChE Fusion Proteins

A cDNA encoding wild type BuChE (positions 1-574 of SEQ ID NO:1) or truncated BuChE (positions 1-530 of SEQ ID NO:1) can be amplified by PCR from a commercially available human cDNA library such as the fetal liver cDNA library (Clonetech). Residues 531-574 of SEQ ID NO:1 from the native BuChE sequence preferably are not present in a final protein construct because this region of the protein is hydrophobic and may cause the recombinant protein to aggregate. The cDNA can be designed to include the BuChE natural signal sequence required for secretion of the proteins from the cell. The cDNAs also are designed to delete the termination codon and add an in-frame unique restriction site to facilitate joining to DNA sequences encoding the IgG C_{H} or Fc domains. The reverse oligo also may encode an amino acid or peptide linker. Alternatively the gene can be modified such that no linker is present as to result in a direct fusion between the BuChE and the IgG proteins. The latter changes are included in the reverse primers used for PCR amplification or in oligonucleotides used to assemble the genes. The forward primers may include an optimized Kozak sequence (GCC(A/G)CCATGG; SEQ ID NO:9), where the underlined ATG is the initiator methionine of the protein) for efficient translation of the proteins in mammalian cells (Kozak, 1991). Site-directed mutagenesis of cysteine residue 400 to a different amino acid such as serine or alanine can be accomplished using PCR mutagenesis as described by Higuchi (1990) or Scharf (1990). The final clones should be verified by sequencing.

The Fc and C_{H} domains of human IgG1 and IgG4 can be cloned as described by Cox et al., 2004 and PCT Publication No. Publication No. WO 01/03737 A1. These fragments can be used to create the IgG-Fc and IgG-C_{H} fusion proteins. Preferred fusion points are alanine at position 1 of the C_{H}1 domain and glutamic acid at the beginning of the Fc domain (Ellison et al., 1981; 1982).

Genes encoding the BuChE fusion proteins can be cloned into a mammalian cell expression vector such as pcDNA3.1, available from Invitrogen, Inc. pcDNA3.1 can be used for both transient transfection and stable transformation of COS and CHO cells. The plasmid contains a polylinker for cloning target genes downstream of the strong cytomegalovirus promoter, an SV40 origin of replication for high copy number replication in COS cells and selectable markers for growth in bacteria (ampicillin resistance) and mammalian cells (G418 resistance). Plasmid DNAs are isolated using commercially available kits (e.g., Qiagen, Inc.) and used to transfect monkey COS cells in vitro. COS cells are plated in T75 tissue culture flasks and transfected the next day with appropriate plasmids using well-established procedures (Bebbington, 1996; Linsley et al., 1991). Following a 24 hr grow-out in serum-containing media, the cells are washed extensively to remove serum (which could interfere with purification of the IgG fusions by affinity chromatography) and grown for up to an additional 9 days in serum-free media. Conditioned media is collected every three days and pooled for purification. Conditioned media can be concentrated and passed through a Protein A affinity column to purify the IgG fusion proteins. Human IgG1 and IgG4 (through the heavy chain constant region) bind tightly to Protein A whereas contaminating, residual bovine IgGs, which may be present due to use of bovine serum for initial cell growth, bind poorly to this resin (Pierce Immunochemical Reagents Catalogue). Bound proteins are eluted from the column with low pH buffer, neutralized with Tris base, and dialyzed, if desired. The IgG fusion proteins can be purified further using other chromatographic methods such as ion exchange, hydrophobic interaction or size exclusion chromatography. Protein concentrations are determined using commercially available protein assay kits (available from Bio-Rad Laboratories). Typical yields of IgG fusion proteins expressed and purified from transfected COS cells using this procedure are in the range of 100-300 μg/300 ml of conditioned media.

The fusion proteins can also be expressed in insect cells as secreted proteins using standard procedures well known in the art. cDNAs encoding the fusion proteins can be cloned into commercially available vectors, e.g., pVL1392 from Invitrogen, Inc. and cotransfected with linearized baculovirus DNA such as Bac-N-Blue™ baculovirus DNA (Invitrogen) into insect cells such as Sf9 cells. Plaques containing recombinant viruses can be isolated, amplified and used to infect insect cells. Infected insect cells are grown for several days. Aliquots of the conditioned media can be collected on a daily basis and analyzed for the presence of the secreted proteins by SDS-PAGE, followed by Western blots using appropriate antisera (Accurate Chemical). Fusion proteins are purified from conditioned media using Protein A affinity chromatography, as described above. Bound proteins are released from the columns using low pH buffer, neutralized and dialyzed. If needed, the proteins can be purified further using other column chromatography procedures including, but not limited to size exclusion and ion exchange column chromatography procedures.

### Example 6

### Pharmacokinetic Experiments with PEGylated BuChE

Pharmacokinetic (PK) studies of PEGylated BuChE are performed to determine to what extent PEGylation lengthens the in vivo half-life of the protein. PK study tests both wild type BuChE and PEGylated BuChE. Three rats receive an intravenous bolus injection (10 μg/kg to 10 mg/kg, preferably 100 μg/kg) of one of the test proteins, and circulating levels of the protein are measured over the course of 120 hr. Blood samples are collected at 0, 0.5, 1.5, 4, 8, 24, 48, 72, 96, and 120 hr following intravenous administration. Protein levels can be quantitated using commercially available human BuChE ELISA kits or Western Blot using a BuChE monoclonal affinity purified antibody (available from Accurate Chemical). Addition PK studies can explore PEG molecules of different sizes (10, 20, and 40 kDa) and different routes of administration (intravenous and subcutaneous) following a similar protocol.

### Example 7

### Pharmacokinetic Experiments with the BuChE Immunofusion Proteins

Extensive pharmacokinetic studies of the fusion proteins can be performed to determine to what extent fusion of the proteins to the Fc or C_{H} domains of human IgGs extends the in vivo half-life of the protein. Groups of three rats receive an intravenous bolus injection of each protein and circulating levels of the proteins are measured over the course of 144 hr. Protein levels can be quantitated using commercially available human ELISA kits for BuChE or by Western blot. Additional experiments can be performed using the subcutaneous route of administration.

### Example 8

### Investigating the Efficacy of Long Acting Forms of BuChE in Animal Toxicity Models

The protective effects of PEG-Cys-BuChE as a enzyme bioscavenger can be evaluated in mice as described by Yuan et al. (1999). Succinylcholine (a muscle relaxant) is used as a surrogate cholinesterase inhibitor in lieu of an OP nerve agent. Mice are given varying amounts of BuChE, PEGylated BuChE, or BuChE immunofusion before being challenged with a 1.5× lethal dose of succinylcholine. Symptoms of intoxication are monitored visually.

### REFERENCES

Abuchowski, A., Kazo, G. M., Verhoest, C. R., Van Es, T., Kafkewitz, D., Nucci, M. L., Viau, A. T. and Davis, F. F. (1984) Cancer Biochem. Biophys. 7: 175-186. Cancer therapy with chemically modified enzymes: antitumor properties of polyethylene glycol-asparaginase.
Allon, N., Raveh, L., Gilat, E., Grunwald, J., and Ashani, Y. (1998) Toxicol. Sci. 43, 121-128. Prophylaxis against soman inhalation toxicity in guinea pigs by pretreatment alone with human serum butyrylcholinesterase.
Altamirano, C., Bartels, C. and Lockridge, O. (2000) J. Neurochem. 74: 869-877. The butyrylcholinesterase K-variant shows similar cellular protein turnover and quaternary interaction to the wild-type enzyme.
Baldassarre H, Wang B, Keefer C L, Lazaris A, and Karatzas C N.(2004) Reprod Fertil Dev. 2004; 16(4):465-70. State of the art in the production of transgenic goats.
Bailon P, Palleroni A, Schaffer C A, Spence C L, Fung W J, Porter J E, Ehrlich G K, Pan W, Xu Z X, Modi M W, Farid A, Berthold W, Graves M. (2001) Bioconjug Chem. 12(2): 195-202. Rational design of a potent, long-lasting form of interferon: a 40 kDa branched polyethylene glycol-conjugated interferon alpha-2a for the treatment of hepatitis C.
Blong, R., Bedows, E. and Lockridge, O. (1997) Biochem. J. 327:747-757. Tetramerization domain of human butyrylcholinesterase is at the C-terminus.
Brandeis R., Raveh L., Grunwald, J., Cohen, E. and Ashani, Y. (1993) Pharmacol Biochem Behav. 46:889-896. Prevention of soman-induced cognitive deficits by pretreatment with human butyrylcholinesterase in rats.
Broomfield, C A, Maxwell, D M, Solana, R P, Castro, C A, Finger, A V and Lenz, D E (1991) J Pharmacol and Exp Therapeut, Vol. 259: 633-638. Protection of butyrylcholinesterase against organophosphorus poisoning in non-human primates.
Capon, D. J., Chamow, S. C., Mordenti, J., Marsters, S. A., Gregory, T., Mitsuya, H., Byrn, R. A., Lucas, C., Wurm, F. M., Groopman, J. E., Broder, S, and Smith, D. H. (1989) Nature 337: 525-531.
Cascio, C, Comite, C, Ghiara, M, Lanza, G. and Ponchione, A. (1998) Minerva Anestesiol (Italian) 54: 337-345. The use of serum cholinesterase in severe phosphorus poisoning,
Cheng, T. Harvey, S. and Stroup, A. (1993) Appl. Environ. Microbial. 59:3138-3140. Purification and properties of a highly active organophosphorus acid anhydrolase from Alteromonas undina.
Chitlaru, T., Kronman, C., Velan, B. and Shafferman, A. (2001) Biochem. J. 354, 613-625. Effect of human acetylcholinesterase subunit assembly on its circulatory residence.
Chitlaru, T., Kronman, C., Velan, B. and Shafferman, A. (2002) Biochem. J. 363, 619-631. Overloading and removal of N-glycosylation targets on human acetylcholinesterase: effects on glycan composition and circulatory residence time.
Costa L G, Li W F, Richter R J, Shih D M, Lusis A, Furlong C E. (1999) Chem Biol Interact. 119-120:429-38. The role of paraoxonase (PON1) in the detoxication of organophosphates and its human polymorphism. Cohen, O., Kronman, C, Chitlaru, T., Ordentlich, A., Velan, B. and Shafferman, A. (2001) Biochem. J. 357:795-802. Effects of chemical modification of recombinant acetylcholinesterase by polyethylene glycol on its circulatory longevity.
Cox, G. N. Smith, D., Carlson, S. Bendele, A., Chlipala, E. and Doherty D. (2004) Exp. Hematol. 32: 441-449. Enhanced circulating half-life and hematopoietic properties of a human GCSF/immunoglobulin fusion protein.
Delgado, C. Francis, G E, and Derek (1992) Critical Rev Ther Drug Carrier Sys 9:249-304. The uses and properties of PEG-linked proteins.
Dumas, D. Caldwell, S. Wild, J and Raushel, F. (1989) J. Biol. Chem. 264:19659-19665. Purification and properties of phosphotriesterase from Pseudomonas diminuta.
Duysen, E., Bartels, C. and Lockridge, O. (2002) J. Pharmacol. and Experimental Therapeutics. 302:751-758. Wild type and A328W Mutant Human BuChE tetramers expressed in Chinese Hamster Ovary Cells have a longer half-life in circulation and protect mice from cocaine toxicity.
Ellison, J., Buxbaum, J. and Hood, L. (1991) DNA 1: 11-18. Nucleotide sequence of a human immunoglobulin C gamma 4 gene.
Ellison, J. W., Berson, B. J. and Hood, L. E. (1982) Nucleic Acids Research 10: 4071-4079. The nucleotide sequence of a human immunoglobulin C gamma 1 gene.
Ellman, G. Courtney, K., Andres, V. and Featherstone, R. (1961) Biochem. Pharmacol. 7: 88-95. A new and rapid colorimetric determination of acetylcholinesterase activity.
Fee, C. (2003) Biotech Bioeng. 82:200-206. Size-exclusion chromatography: A new technique for protein PEGylation.
Fisher, M., Ittah, A. Gorecki, M., and Weber, M. (1995) Biotechnol Appl. Biochem. 21:295-311. Recombinant human acetylcholinesterase expressed in E. coli: refolding, purification and characterization.
Foser S, Schacher A, Weyer K A, Brugger D, Dietel E, Marti S, Schreitmuller T. (2003) Protein Expr Purif. 30(1):78-87. Isolation, structural characterization, and antiviral activity of positional isomers of monopegylated interferon alpha-2a (PEGASYS).
Goldman, M. (2003) BioProcess International 1:9. 60-63. Processing Challenges for Transgenic Milk Products
Goodson, R. J. and Katre, N. V. (1990) Biotechnology 8: 343-346. Site-directed pegylation of recombinant interleukin-2 at its glycosylation site.
Harris, J. M., Martin N. E. and Modi, M. (2001) Clin. Pharmacokinet. 40:539-551. PEGylation: A novel process for modifying pharmacokinetics.
Harris, J. M. and Chess, R. B. (2003) Nat. Rev. Drug Discov 2:214-221. Effect of PEGylation on pharmaceuticals.
Hershfield, M. S., Buckley, R. H., Greenberg, M. L. et al., (1987) N. Engl. J. Medicine 316: 589-596. Treatment of adenosine deaminase deficiency with polyethylene glycol-modified adenosine deaminase.
Higuchi, R. in “PCR Protocols” (M A Innis, D H Gelfand, J J Sninsky, & T J White, eds.) Academic Press
Hooftman, G. Herman, S., Schacht, E. (1996) J. Bioactive Compatible Polymer 11:135-139. PEGs with reactive endgroups II. Practical considerations for the preparation of protein-PEG conjugates.
Katre, N. V. (1990) J. Immunology 144: 209-213. Immunogenicity of recombinant IL-2 modified by covalent attachment of polyethylene glycol.
Katre, N. V., Knauf, M. J. and Laird, W. J. (1987) Proc. Natl. Acad. Sci. USA 84: 1487-1491. Chemical modification of recombinant interleukin-2 by polyethylene glycol increases its potency in the murine sarcoma model.
Keating, M. J., Holmes, R., Lerner, S. and Ho, D. H. (1993) Leuk. Lymphoma 10, 153. L-asparaginase and PEG asparaginase—past, present, and future.
Kozak, M. (1991) J. Biol. Chem. 266: 19867-19870. Structural features in eukaryotic mRNAs that modulate the initiation of translation.
Kronman, C., Chitlaru, T., Elhanany, E., Velan, B. and Shafferman, A. (2000) J. Biol. Chem. 275, 29488-29502. Hierarchy of post-translation modifications involved in the circulatory longevity of glycoproteins: demonstration of concerted contributions of glycan sialylation and subunit assembly to the pharmacokinetic behaviour of bovine acetylcholinesterase.
Kronman, C., Velan, B., Marcus, D., Ordentlich, A., Reuveny, S. and Shafferman, A. (1995) Biochem. J. 311: 959-967. Involvement of oligomerization, N-glycosylation and sialylation in the clearance of cholinesterases from the circulation.
Linsley, P. S., Brady, W., Urnes, M., Grosmaire, L. S., Damle, N. K. and Ledbetter, J. A. (1991) J. Exp. Med. 174: 561-569. CTLA-4 is a second receptor for the B cell activation antigen B7.
Lockridge, O., Bartels, C., Vaughan, T., Wong, C., Norton, S. and Johnson, L (1987) J. Biol. Chem., 262: 549-557. Complete amino acid sequence of human serum cholinesterase.
Lockridge, O., Adkins, S. and La Du, B. (1987) J. Biol. Chem., 262:12945-12952. Location of disulfide bonds within the sequence of human serum cholinesterase.
Lockridge, O., Blong, R. M. Masson, P., Froment, M. Millard, C. and Broomfield, C. (1997) Biochemistry 36:786-795. A single amino acid substitution Gly117His confers phosphotriesterase organophosphorus acid anhydride hydrolase activity on human butyrylcholinesterase.
Masson, P. (1990) “Expression and Refolding of Functional Human BuChE from E. coli,” Multiple Approaches to Cholinesterase Functions (Eds. Shafferaman, A. and Velan, B.), Plenum, N.Y., pp. 49-52
Masson, P., Adkins, S., Gouet, P. and Lockridge, O. (1993) J Biol Chem 268:14329-14341. Recombinant human butyrylcholinesterase G390V, the fluoride-2 variant, expressed in CHO cells, is a low affinity variant.
Meyers, F. J., Paradise, C., Scudder, S. A., Goodman, G., and Konrad, M. (1991) Clin Pharmacol Ther 49:307-313. A phase I study including pharmacokinetics of polyethylene glycol conjugated interleukin-2.
Monkarsh, S P, Ma, Y, Aglione, A, Bailon, P, Ciolek, D, DeBarbieri, B, Graves, M C, Hollfelder, K, Michel, H, Palleroni A, Porter, J E, Russoman, E, Roy, S. and Pan Y C. (1997) Anal Biochem. 247(2):434-440. Positional isomers of monopegylated interferon alpha: isolation, characterization, and biological activity.
Morpurgo, M. and Veronese, F. (2004) in Methods in Molecular Biology 283: 45-70. Conjugates of Peptides and Proteins to Polyethylene Glycols.
Nachon, F., Nicolet, Y., Viguie, N., Masson, P., Fontecilla-Camps, J. C., and Lockridge, O. (2002) Eur. J. Biochem. 269, 630-637. Engineering of a monomeric and low-glycosylated form of human butyrylcholinesterase: expression, purification, characterization and crystallization.
Nicolet, Y, Lockridge, O., Masson, P., Fontecilla-Camps, J., and Nachon, F. (2003) J. Biol. Chem. 278:41141-41147. Crystal structure of human butyrylcholinesterase and of its complexes with substrate and products.
Platteborze, P. and Broomfield, C. (2002) Biotechnol Appl Biochem. (2000) 31:225-229. Expression of biologically active human butyrylcholinesterase in cabbage looper (Trichoplusia ni).
Raveh, L, Grauer, E, Grunwald, J, Cohen, E and Ashani, Y. (1997) Toxicol. Appl Pharmacol. 145: 43-53. The stoichiometry of Protection against soman and VX toxicity in monkeys pretreated with human BuChe.
Raveh, L., Grunwald, J., Marcus, D., Papier, Y., Cohen, E., and Ashani, Y. (1993) Biochem. Pharmacol. 45, 2465-2474. Human butyrylcholinesterase as a general prophylactic antidote for nerve agent toxicity. In vitro and in vivo quantitative characterization.
Raveh L., Ashani, Y., Levy, D., De La Hoz, D., Wolfe, A. D. and Doctor, B. P. (1989) Biochem Pharmacol. 38: 529-534. Acetylcholinesterase prophylaxis against organophosphate poisoning. Quantitative correlation between protection and blood-enzyme level in mice.
Roitt, I., Brostoff, J. and Male, D. eds. (1989) “Immunology” (Gower Medical Publishing, London, U. K.; New York, N. Y.).
Rosendahl, M., Doherty, D. and Cox, G. N. (2001) WO0187925A2. Methods for folding proteins containing free cysteine residues
Saxena, A., Redman, A. M. G., Jiang, X. L., Lockridge, O., and Doctor, B. P. (1997a) Biochemistry 36: 642-14651. Differences in active site gorge dimensions of cholinesterases revealed by binding of inhibitors to human butyrylcholinesterase.
Saxena, A., Maxwell, D. Quinn, D. Radic, Z., Taylor, P, and Doctor, B. (1997b) Biochem Pharmacol. 54:269-274. Mutant acetylcholinesterases as potential detoxification agents for organophosphate poisoning.
Saxena, A. Ashani, Y., Raveh, L. Stevenson, D., Patel, T. and Doctor, B. P. (1998) Mol. Pharmac. 53: 112-122. Role of oligosaccharides in the pharmacokinetics of tissue derived and genetically engineered cholinesterases.
Schwarz M, Glick D, Loewenstein Y, Soreq H. (1995) Pharmacol Ther. 67:283-322. Engineering of human cholinesterases explains and predicts diverse consequences of administration of various drugs and poisons.
Scharf, S. J. in “PCR Protocols” (M A Innis, D H Gelfand, J J Sninsky, & T J White, eds.) Academic Press (1990) pp. 84-91 “Cloning with PCR”
Sheffield, W. P. (2001) Current Drug Targets 1:1-10. Modification of clearance of therapeutic and potentially therapeutic proteins.
Sims, G. and Snap, T. (1980) Anal. Biochem. 107:60-63. A method for the estimation of polyethylene glycol in plasma protein fractions.
Soreq, H., Seidman, S., Dreyfus, P. A., Zevin-Sonkin, D. and Zakut, H. (1989) J. Biol. Chem. 264:10608-10613. Expression and tissue-specific assembly of human BuChe in microinjected Xenopus laevis oocytes.
Soreq, H, Zevin-Sonkin D and Razon N. (1984) EMBO Journal 3(6):1371-1375. Expression of cholinesterase gene(s) in human brain tissues: translational evidence for multiple mRNA species.
Sun, H., Shen, M. Pang, Y, Lockridge, O. and Brimijoin, S. 2002 J Pharmacol Exp Ther 302: 710-716. Cocaine hydrolase engineered from human BuChE accelerates cocaine metabolism in rats.
Wei, W. L., Qin, J. C. and Sun, M. J. (2000) Biochem. Pharmacol 60:121-126. High-level expression of human BuChe gene in Bombyx mori and biochemical-pharmacological characteristic study of its product.
Sussman, J. L., Harel, M., Frolow, F., Oefner, C., Goldman, A., Toker, L., and Silman, I. (1991) Science 253:872-879. Exogenous administration of plasma-derived ChEs such as AchE from fetal bovine serum and BuchE from equine and human serum have been successfully used as an efficacious prophylatic treatment to prevent poisoning by OP compounds, in both rodent and non-human primate models.
Wolfe, A. D., Blick, D. W., Murphy, M. R., Miller, S. A., Gentry, M. K., Hartgraves, S. L. and Doctor, B. P. (1992) Toxicol. Appl Pharmacol., 117: 189-193. Use of cholinesterases as pretreatment drugs for the protection of rhesus monkeys against soman toxicity.
Yuan, H., Yu, W, She, C, and Sun, M. (1999) Zhongguo Yao Li Xue Bao. 20:74-80. Characteristics of recombinant
Zalispky, C. (1995) Adv. Drug Delivery Rev 16:157-182.
Zhan, C. G., Zheng, F. and Landry, D. W. (2003) J Am Chem. Soc. 2003 Mar. 5; 125(9):2462-74. Fundamental reaction mechanism for cocaine hydrolysis in human butyrylcholinesterase.
U.S. Provisional Patent Application Ser. No. 60/633,745, filed Dec. 6, 2004

Each publication cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth above and in the following claims.

1. An isolated PEGylated butyrylcholinesterase protein, comprising at least one polyethylene glycol attached to at least one amino acid of a butyrylcholinesterase protein.

2. The PEGylated butyrylcholinesterase protein of claim 1, wherein a polyethylene glycol is attached to at least one lysine or cysteine residue in said butyrylcholinesterase protein.

3. The PEGylated butyrylcholinesterase protein of claim 2, wherein the cysteine residue is selected from the group consisting of: Cys66 and Cys571, with respect to SEQ ID NO:1.

4. The PEGylated butyrylcholinesterase protein of claim 2, wherein the lysine residue is selected from the group consisting of: K9, K12, K44, K45, K51, K103, K105, K131, K180, K248, K262, K313, K 314, K323, K348, K407, K408, K458, K469, K476, K494, and K513, with respect to SEQ ID NO:1.

5. The PEGylated butyrylcholinesterase protein of claim 1, wherein a polyethylene glycol is attached to the amino-terminal amino acid of said butyrylcholinesterase protein.

6. The PEGylated butyrylcholinesterase protein of claim 1, wherein said polyethylene glycol is attached to at least one added thiol in said butyrylcholinesterase protein.

7. The PEGylated butyrylcholinesterase protein of claim 6, wherein said added thiol is attached to at least one amino group in said butyrylcholinesterase protein.

8. The PEGylated butyrylcholinesterase protein of claim 6, wherein said added thiol is attached to at least one lysine residue in said butyrylcholinesterase protein.

9. The PEGylated butyrylcholinesterase protein of claim 6, wherein said added thiol is attached to the amino-terminal amino acid of said butyrylcholinesterase protein.

10. The PEGylated butyrylcholinesterase protein of claim 1, wherein a polyethylene glycol is attached to at least one cysteine residue substituted for at least one amino acid in said butyrylcholinesterase protein.

11. The PEGylated butyrylcholinesterase protein of claim 10, wherein a polyethylene glycol is attached to at least one cysteine residue substituted for at least one amino acid selected from the group consisting of: asparagine-17, asparagine-57, asparagine-106, asparagine-241, asparagine-256, asparagine-341, asparagine-455, asparagine-481, and asparagine-486 of said butyrylcholinesterase protein, with respect to SEQ ID NO:1.

12. The PEGylated butyrylcholinesterase protein of claim 10, wherein a polyethylene glycol is attached to at least one cysteine residue substituted for at least one amino acid selected from the group consisting of: E1, D2, D3, I4, I5, I6, A7, T8, K9, N10, G11, K12, R14, G15, M16, N17, L18, and T19, with respect to SEQ ID NO:1.

13. The PEGylated butyrylcholinesterase protein of claim 10, wherein a polyethylene glycol is attached to at least one cysteine residue substituted for at least one amino acid selected from the group consisting of: R520, T523, S524, F525, P527, K528, V529, and any of positions 530 to 574, with respect to SEQ ID NO:1.

14. The PEGylated butyrylcholinesterase protein of claim 10, wherein a polyethylene glycol is attached to at least one cysteine residue substituted for at least one amino acid in the first 7 residues of said butyrylcholinesterase protein or in the last 50 residues of said butyrylcholinesterase protein.

15. The PEGylated butyrylcholinesterase protein of claim 1, wherein a polyethylene glycol is attached to at least one cysteine residue added preceding the first or following the last amino acid of the mature form of said butyrylcholinesterase protein.

16. The PEGylated butyrylcholinesterase protein of claim 1, wherein the butyrylcholinesterase protein is truncated after position 530, with respect to SEQ ID NO:1.

17. The PEGylated butyrylcholinesterase protein of claim 1, wherein the butyrylcholinesterase protein comprises at least one mutation that enhances the catalytic activity of the butyrylcholinesterase, wherein the mutation is at an amino acid position selected from the group consisting of: W82, W112, G117, Q119, Y128, E197, S198, A199, W231, L277, L286, L298, V288, E325, A328, F329, V331, W430, H438, and Y440.

18. The PEGylated butyrylcholinesterase protein of claim 1, wherein the butyrylcholinesterase protein comprises an amino acid sequence that is at least 95% identical to SEQ ID NO:1, wherein said protein catalyzes the hydrolysis of a carboxylester.

19. The PEGylated butyrylcholinesterase protein of claim 1, wherein the butyrylcholinesterase protein is covalently joined to a non-butyrylcholinesterase protein.

20. A fusion protein comprising a butyrylcholinesterase protein covalently joined to a non-butyrylcholinesterase protein.

21. The fusion protein of claim 20, wherein said fusion protein comprises butyrylcholinesterase covalently joined to an immunoglobulin (Ig) domain.

22. The fusion protein of claim 21, wherein the immunoglobulin domain is selected from the group consisting of IgG-Fc, IgG-C_{H} and IgG-C_{L}.

23. The fusion protein of claim 20, wherein said fusion protein is dimeric.

24. An isolated nucleic acid molecule encoding the protein of claim 1.

25. A recombinant nucleic acid molecule comprising the nucleic acid molecule of claim 24, operatively linked to at least one expression control sequence.

26. A recombinant host cell that expresses the recombinant nucleic acid molecule of claim 25.

27. The recombinant host cell of claim 26, wherein said host cell is selected from the group consisting of a bacterium, a yeast, a mammalian cell, an insect cell, and a plant cell.

28. A non-human organism that has been genetically modified to express the recombinant nucleic acid molecule of claim 25.

29. The non-human organism of claim 28, wherein said non-human organism is selected from the group consisting of a plant and an animal.

30. A method to detoxify a carboxylester compound, comprising contacting the compound with the PEGylated protein according to claim 1.

31. A method to detoxify a carboxylester compound, comprising contacting the compound with the fusion protein according to claim 1.


SEQUENCE LISTING (11 sequences)

SEQ ID NO: 1 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 2 - protein, 583 aa, Homo sapiens
EGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 3 - DNA, 36 bp, Artificial
  oligonucleotide primer
cgccatatgggatccatcttggaggatgattaaatg

SEQ ID NO: 4 - DNA, 33 bp, Artificial
  oligonucleotide primer
ttggaggatgattaaatggaagatgacatcatt

SEQ ID NO: 5 - DNA, 40 bp, Artificial
  oligonucleotide primer
atggaagatgacatcattattgcaacaaagaatggtaaag

SEQ ID NO: 6 - DNA, 39 bp, Artificial
  oligonucleotide primer
tgcaacaaagaatggtaaagtccgtgggatgaacttgac

SEQ ID NO: 7 - DNA, 39 bp, Artificial
  oligonucleotide primer
cggggtaccttacaagacttttggaaaaaatgatgtcca

SEQ ID NO: 8 - DNA, 22 bp, Artificial
  oligonucleotide primer
caacaaatgcttcattcagaag

SEQ ID NO: 9 - DNA, 10 bp, Artificial
  Kozak Sequence
gccrccatgg

SEQ ID NO: 10 - DNA, 2444 bp, Homo sapiens
  CDS (158)..(1966)
agtaacagttgattgttacattcagtaacactgaatgtcagtgcagtccaatttacaggctggagcagcagctgcatcctgcatttccccgaagtattacatgattttcactccttgcaaactttaccatctttgttgcagagaatcggaaatcaatatgcatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggatcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacatgtttaattaaatttcacaatataaagttctacagttaattatgtgcatattaaaacaatggcctggttcaatttctttctttccttaataaatttaagttttttccccccaaaattatcagtgctctgcttttagtcacgtgtattttcattaccactcgtaaaaaggtatcttttttaaatgaattaaatattgaaacactgtacaccatagtttacaatattatgtttcctaattaaaataagaattgaatgtcaatatgagatattaaaataagcacagaaaatc

SEQ ID NO: 11 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL